(19) United States
(12) Patent Application Publication
(10) Pub. No.: US 2024/0387036 A1
(43) Pub. Date: Nov. 21, 2024

(54) INTELLIGENT RULES BASED METHODOLOGY AND SYSTEM TO PROVIDE PREDICTIVE PATIENT STATUS MONITORING AND ALERTS

(71) Applicant: Javier Vinals, Irving, TX (US)

(72) Inventor: Javier Vinals, Irving, TX (US)

(21) Appl. No.: 18/199,385

(22) Filed: May 19, 2023

(51) Int. Cl. G16H 40/67

(52) U.S. Cl. CPC G16H 40/67

(57) ABSTRACT

This invention relates in general to a physician directed chronic care patient care and measurement reporting Electronic Health Records (EHR) systems and supporting applications which enable structured online caregiver to patient interaction.

### REFERENCE TO RELATED PATENT APPLICATIONS

This application claims the benefit of U.S. Provisional Pat. App. Ser. No. 62/528,852, filed on Jul. 5, 2017, entitled, “Interactive Template Driven System to Support Physician Directed Chronic Care Plan Management” of which certain core functions are referenced herein as an “online caregiver and patient engagement network” functioning as an overall patient relationship management system.

### REFERENCES (U.S. Patent)

| | | |
|---|---|---|
| 6,151,581 | November 2000 | Kraftson |
| 8,734,428 | May 2014 | Blomquist |
| 2008/0281168 | November 2008 | Gibson |
| 2009/0216556 | August 2009 | Martin |
| 2010/0057646 | March 2010 | Martin |
| 2011/0119075 | May 2011 | Dhoble |
| 2014/0180711 | June 2014 | Kamen |

### OTHER REFERENCES

a. “Using Computerized Patient-Alert Systems” April, 2013, Fenwick, Leaman A., Princess Royal Hospital, UK

b. “A Strong Pulse for Biometrics in Healthcare”, (PlanetBioMetrics.Com, 2013 September 27)

c. “Knowledge Management in Health Care: an Architectural Framework for Clinical Process Management Systems”, DEXA, IEEE Computer Society, 2005

### DESCRIPTION

### Field of the Invention

This invention relates in general to a physician directed chronic care patient care and measurement reporting Electronic Health Records (EHR) systems and supporting applications which enable structured online caregiver to patient interaction. In particular, the proposed methodology and system provides active intelligent monitoring of patient status via active parsing of patient activities, records and caregiver input to provide predictive and reactive alert notifications that initiate caregiver intervention based on the alert information.

### BACKGROUND OF THE INVENTION

Chronic Care Management (CCM) is defined as the non-face-to-face services provided to Medicare beneficiaries who have two or more significant chronic conditions. In addition to office visits and other face-to face encounters, which must be billed separately, these services include communication with the patient and other treating health professionals for care coordination (both electronically and by phone), medication management, and providing accessibility 24 hours a day to patients and other treating physicians or clinical staff. The creation and revision of electronic care plans is also a key component of CCM.

Predictive “active” analysis of patient record data and status is a tool that will improve successful patient outcomes and enhance caregiver efficiency. The present invention proposes a methodology which can be incorporated into the process flows for a plurality of healthcare systems which provide a functional caregiver to patient service delivery monitoring capability and supporting verification and assurance means to provide documented (auditable) evidence of the appropriate caregiver intervention relating to predictive intervention alerts.

### BRIEF DESCRIPTION OF THE DRAWINGS

FIG. 1: is a system block diagram of a typical physician to patient electronic health record access system and associated EMR subsystems.

FIG. 2: illustrates the methodology and flow for the patient monitoring and alert service.

FIG. 3: illustrates an example of the system template and dashboard with a patient triggered event alert.

### DETAILED DESCRIPTION OF THE PREFERRED EMBODIMENT

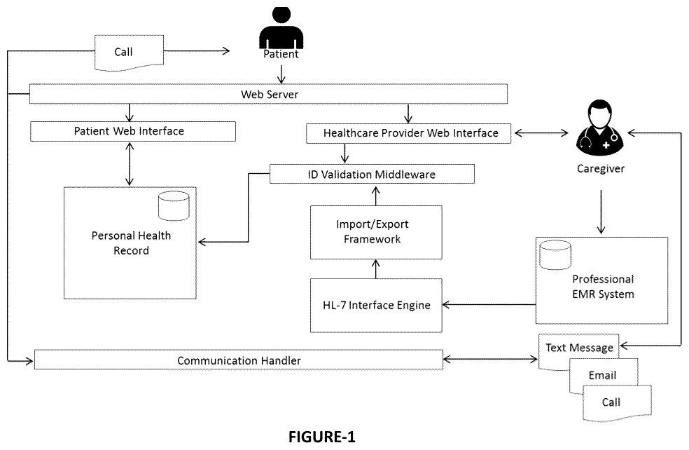

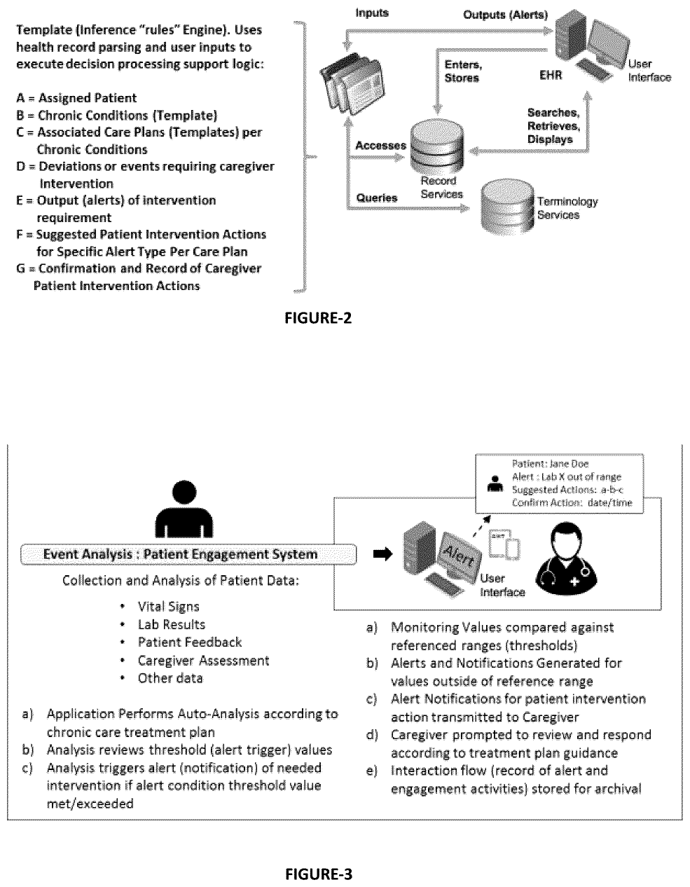

An online caregiver and patient engagement network system (FIG. 1) that is configured to: 1) Provide active verification of authorized caregiver access. 2) Enable caregiver to select a specific CCM patient for review or view a plurality of patients in a single dashboard view. 3) Intelligent system parsing engine that identifies conditional threshold deviations or activities for a patient that require caregiver intervention (FIG. 2) and issues alert notifications (FIG. 3) to the assigned caregiver for a specific intervention action. 4) A timed validation record (FIG. 2) is initiated and maintained for the alert and a record of the caregiver intervention activity for the specific alert is stored for verification and archival reporting to the main caregiver system.

What is claimed is:

1. An online caregiver and chronic care patient engagement system with corresponding methodology that uses intelligent rules based logic analysis and visual dashboard technology to proactively monitor patient status and issue alert notifications when general or specific intervention for a patient by physician and/or caregivers is required.
The interactive chronic care plan engagement system of claim 1 wherein the assigned caregiver must authenticate their permission level and login to the system to access patient application data and engagement platform.
The interactive chronic care plan engagement system and application of claim 1 wherein the caregiver can select an individual patient in the Chronic Care system for direct Care Plan interaction.
The interactive chronic care plan engagement system and application of claim 1, wherein the caregiver has a dashboard depicting patient status for a single or plurality of assigned patients.
The interactive chronic care plan engagement application system of claim 1, wherein the system actively monitors patient data and detects deviations, noncompliance or scheduled events that require caregiver intervention.
The interactive chronic care plan engagement system of claim 1, wherein upon system recognition of an event requirement necessitating “patient intervention” the system issues an alert and notification to the caregiver via the dashboard and a plurality of contact methods (phone, text, email).
The interactive chronic care plan engagement system of claim 1, wherein upon system alert and notification to the caregiver via the dashboard and a plurality of contact methods (phone, text, email) the caregiver must confirm “intervention” action is taken and that action is recorded (archived) into the system storage.
